(19) United States
(12) Patent Application Publication
(10) Pub. No.: US 2016/0106940 A1
(43) Pub. Date: Apr. 21, 2016

(54) TUBE TIP BEVEL TO AID WIRE GUIDE INSERTION

(71) Applicant: Cook Medical Technologies LLC, Bloomington, IN (US)

(72) Inventor: Christopher D. Bosel, Bloomington, IN (US)

(21) Appl. No.: 14/880,860

(22) Filed: Oct. 12, 2015

Related U.S. Application Data
(60) Provisional application No. 62/064,163, filed on Oct. 15, 2014.

(51) Int. Cl. A61M 16/04

(52) U.S. Cl. CPC A61M 16/0472; A61M 16/0488

(57) ABSTRACT

A dilator assembly is provided having a curved dilator being defined by a concave and a convex side and having at least one lumen. The dilator assembly also has a guiding catheter with at least one lumen, and the guiding catheter is disposed within the lumen of the curved dilator. The guiding catheter also has a bevel on the end of the catheter that facilitates insertion of a guide wire into the lumen of the catheter. Additionally, the bevel is oriented towards the convex side of the curved dilator to prevent the guide wire from separating from the guiding catheter. The bevel may also be designed to maximize the ease of feeding the guide wire into the lumen of the catheter while also maintaining the catheter's shape and rigidity.

### FIELD

The present disclosure relates generally to medical devices and more particularly to catheters used in conjunction with guide wires. Additionally, the present disclosure relates to tracheostomy procedures.

### BACKGROUND

The statements in this section merely provide background information related to the present disclosure and may not constitute prior art.

Catheters are medical devices that are used in a wide range of medical applications such as for cardiovascular, urological, gastrointestinal, and other uses. They are usually inserted into the body through a cavity, either a natural cavity or one surgically created. Catheters are generally used for fluid exchange or to provide access into the body for surgical instruments. Catheters are usually made of a flexible material and are tubular in shape with at least one lumen, which is a tube that runs through the catheter.

Often, a guide wire is used in conjunction with the catheter. A guide wire is a long, flexible wire that is inserted into the body before the catheter. The catheter is then fed over the guide wire by inserting the guide wire into one of the catheter's lumens and then sliding the catheter down the length of the guide wire until the catheter is in the desired position. Frequently, the guide wire is then removed, leaving the catheter in place.

Ideally, the guide wire is designed to fit in the catheter's lumen with a minimal annular clearance between the outer diameter of the guide wire and the inner diameter of the catheter, which prevents radial movement of the guide wire within the catheter. The minimal clearance allows the clinician to smoothly feed the catheter over the guide wire without risk of the guide wire and catheter separating or getting caught. However, this minimal clearance creates difficulties for clinicians when they are attempting to initially feed the guide wire into the catheter because the guide wire must be perfectly aligned with the catheter's lumen. These difficulties can result in a longer procedure, potentially wasting valuable time. Thus, there is a need to improve the design of catheters to ease the insertion of a guide wire into a catheter.

A tracheostomy is one example of an application where catheters and guide wires are used. A tracheostomy is a surgical procedure that creates an air passage through a patient's neck into the trachea. A tracheostomy is performed when a patient has difficulty breathing without assistance, either due to an obstruction in the normal airway or due to a general inability to breathe independently. A ventilator can then be used to assist the patient in breathing if necessary. If the tracheostomy is required due to an obstruction in the normal airway, a ventilator is generally not required. Often a tracheostomy is performed as a long term or even permanent solution to a patient's inability to breathe independently. In these cases, a tracheal tube is preferable to an endotracheal tube (a tube inserted through the patient's mouth) for patient comfort and for ease of treatment. Endotracheal tubes often require stays in Intensive Care Units and constant patient monitoring, whereas patients with tracheostomy tubes require minimal or even no monitoring. During tracheostomies, clinicians often have difficulty feeding the catheter onto the guide wire, and thus there is a particular need for a catheter design improvement in this specific application.

### SUMMARY

In one form of the present disclosure, a dilator assembly is shown. The dilator assembly comprises a curved dilator having proximal and distal ends and a fixed curvature that is defined by a concave and a convex side. The curved dilator also has at least one lumen curved along a portion of the curved dilator. The dilator assembly further comprises a guiding catheter that comprises a proximal and distal end and at least one lumen. The guiding catheter is disposed within the at least one lumen of the curved dilator and is curved along the curved portion of the at least one lumen of the curved dilator, and the distal end of the catheter extends past the distal end of the curved dilator. The guiding catheter also comprises a bevel on the distal end of the guiding catheter, and the bevel exposes part of the at least one lumen of the guiding catheter at the distal end of the guiding catheter. This bevel is oriented so that the exposed area of the at least one lumen of the guiding catheter faces toward the convex side of the curved dilator. The dilator assembly also has a guide wire comprising a proximal end and a distal end, where the guide wire is disposed within the at least one lumen of the guiding catheter and is curved along the curved portion of the at least one lumen of the curved dilator. Additionally, the distal end of the guide wire extends past the distal end of the guiding catheter. In a more specific form of the present disclosure, the curved dilator is a tracheostomy dilator. In yet another embodiment of the invention, the curved dilator is tapered, with a smaller profile near the distal end of the curved dilator and a larger profile away from the distal end.

In still another form of the present disclosure, a catheter system is provided that comprises a catheter having at least one lumen and a guide wire oriented within the at least one lumen of the catheter. The catheter further comprises a wall surrounding the at least one lumen, an outer diameter extending along a majority of a distal portion of the catheter, an outer surface, and a bevel on at least one end of the catheter. The bevel comprises a distal end, a proximal end, and an intermediate point, where the distal end of the bevel is in line with the at least one end of the catheter. The bevel further comprises a first portion that extends from the distal end to the intermediate point and a second portion that extends from the intermediate point to the proximal end. Along the first portion, the wall of the catheter extends 160-225 degrees circumferentially around the at least one lumen of the catheter without extending around a remaining circumferential area of the at least one lumen. Along the second portion, the wall gradually slopes from the intermediate point to a uniform outer circumference of the catheter at the proximal end of the bevel. Further, the longitudinal distance of the first portion is between 1 and 5 times the outer diameter of the catheter. In another embodiment, the longitudinal distance of the second portion of the bevel is between 1 and 5 times the outer diameter of the catheter.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is an orthographic view of a standard catheter and guide wire assembly;

FIG. 2 is an orthographic view of a catheter guide wire assembly having a bevel;

FIG. 3 is a side view of several different embodiments of the improved bevel design constructed in accordance with the teachings of the present disclosure;

FIG. 4 is an orthographic view of the improved bevel design;

FIG. 5 is a cross section view of the catheter and guide wire assembly;

FIG. 6 is a pictorial representation of a step performed during a tracheostomy procedure;

FIG. 7 is an additional pictorial representation of a step performed during a tracheostomy procedure;

FIG. 8 is an additional pictorial representation of a step performed during a tracheostomy procedure;

FIG. 9 is an additional pictorial representation of a step performed during a tracheostomy procedure;

FIG. 10 is an additional pictorial representation of a step performed during a tracheostomy procedure; and

FIG. 11 is a detailed view of a catheter bevel in a tracheostomy procedure.

### DETAILED DESCRIPTION

The following description is merely exemplary in nature and is not intended to limit the present disclosure, application, or uses. It should be understood that throughout the drawings, corresponding reference numerals indicate like or corresponding parts and features. It should also be understood that various cross-hatching patterns used in the drawings are not intended to limit the specific materials that may be employed with the present disclosure. The cross-hatching patterns are merely exemplary of preferable materials or are used to distinguish between adjacent or mating components illustrated within the drawings for purposes of clarity.

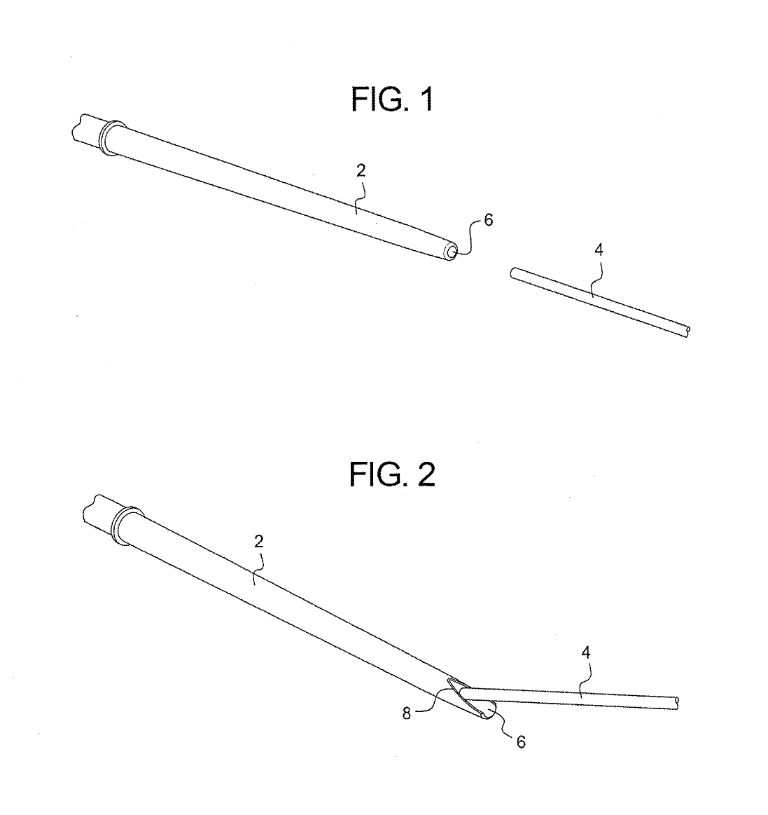

Referring to FIG. 1, a catheter 2 is shown with a guide wire 4. As can be seen, the guide wire 4 must be perfectly aligned with the lumen 6 of the catheter 2 to insert the guide wire 4 into the catheter 2. FIG. 2 shows an improved version of the catheter 2 with a bevel 8. The bevel 8 creates a larger opening of the lumen 6 to allow easier insertion of the guide wire 4. Additionally, the guide wire 4 can now be inserted into the lumen 6 at a slight angle, rather than having to align the guide wire 4 with the lumen 6. However, the bevel shown in FIG. 2 may not be the most preferred design to increase the ease of inserting a guide wire into a catheter. In this design, the bevel 8 and the corresponding opening will only provide a slightly larger opening of the lumen 6 since the bevel 8 narrows immediately from the end of the catheter 2. Thus, guide wire insertion is only minimally simplified.

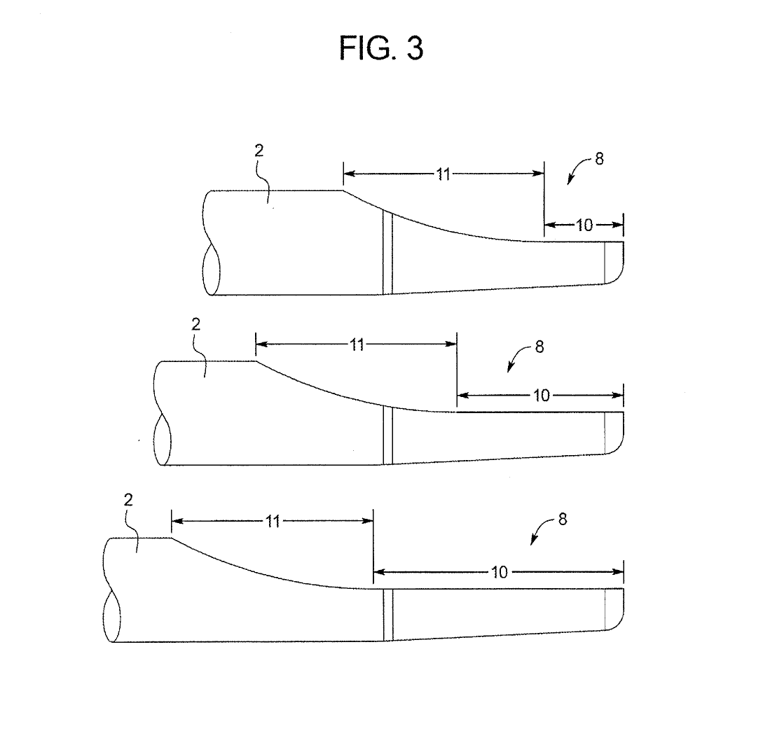

In another embodiment of this invention, the bevel is improved to avoid the issues discussed above. FIG. 3 shows three embodiments of the invention. Here, the bevel 8 need not be a mere angled cut at the end of the catheter 2. Instead, the bevel 8 may begin at the end of the catheter 2 and may have a portion that remains open at a substantially constant angle for a given first length 10, and then the bevel 8 gradually slopes towards the outside of the catheter 2 for a given second length 11 to the end of the bevel 8.

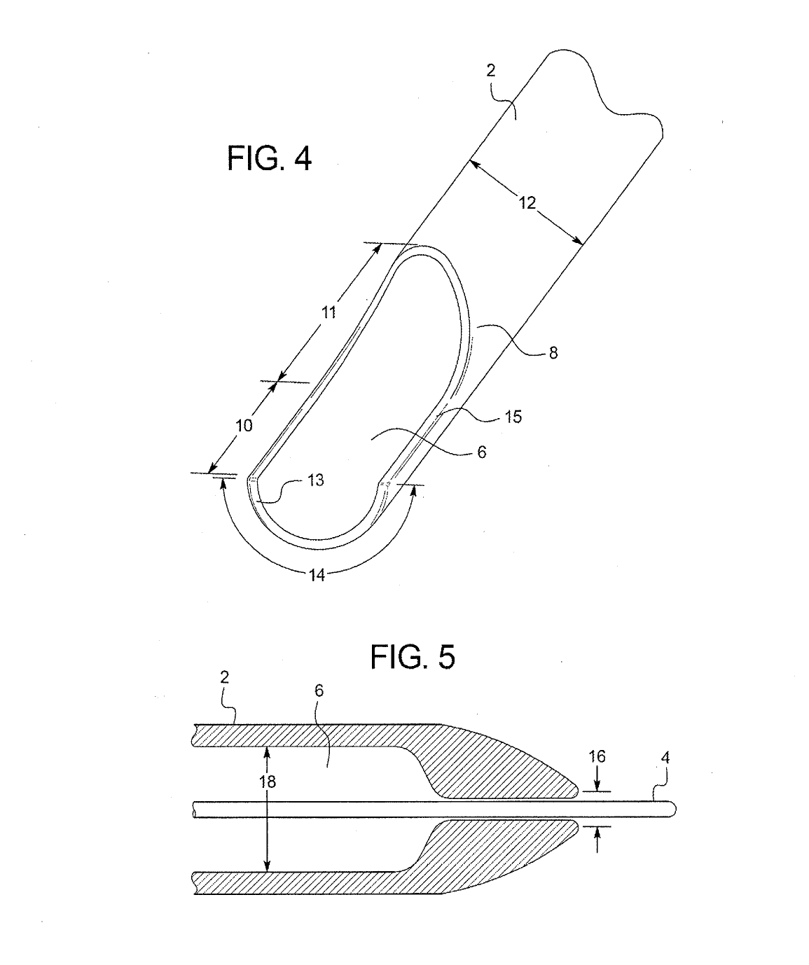

FIG. 4 shows an orthographic view of the end of the catheter 2 and bevel 8. In FIG. 4, the catheter 2 has an outer diameter 12 and a wall 13 surrounding the lumen 6, and the bevel 8 is defined by an angle 14 at the distal end that the wall 13 extends circumferentially around the catheter 2. The angle 14 ideally ranges from 160-225 degrees, so that the end of the catheter 2 retains enough rigidity to keep its shape while also ensuring a sufficient opening of the lumen 6 to fit the guide wire 4, although this range can be adjusted. The angle 14 of the bevel 8 remains substantially constant along the first length 10, which creates a larger opening for the guide wire 4 to be inserted into the lumen 6 of the catheter 2. Alternatively, the bevel 8 may gradually slope outward along the first length as long as the angle 14 remains within the range of 160-225 degrees. At the end of the first length 10, the wall 13 gradually slopes outward along the second length 11 to the end of the bevel 8 where the lumen 6 is completely surrounded by the wall 13 of the catheter 2 and the outer surface of the catheter 2 forms a uniform outer circumference. The first length 10 of the bevel 8 may be determined by a ratio of the first length 10 to the outer diameter 12 of the catheter 2. This first length 10 is chosen to balance the ease of inserting the guide wire 4 into the catheter 2 and the required rigidity of the end of the catheter 2. The first length 10 can be one to five times larger than the outer diameter 12 of the catheter 2, although the first length 10 will ideally be one to three times larger than the outer diameter 12. The second length 11 may be chosen in the same manner as the first length 10. The second length 11 can be one to five times larger than the outer diameter 12, but will ideally be one to two times larger.

Still referring to FIGS. 3-4, the bevel 8 along the second length 11 ideally has a concave arc shape to ensure that the catheter 2 does not catch on anything or cause injury to the patient. For this same reason, the outer edge 15 of the bevel 8 ideally has a fillet along the first length 10 and second length 11 of the bevel 8.

Often, catheters are designed with varying lumen diameters. Referring to FIG. 5, the lumen 6 has two diameters: an end diameter 16 and a middle diameter 18. The lumen 6 is often manufactured with a constant middle diameter 18 so that the catheter 2 can be adapted to a variety of uses. The end diameter 16 may then be altered for specific applications. In this situation, the end diameter 16 is smaller than the middle diameter 18 to ensure a minimal clearance fit with the guide wire 4. Typically, the length of the end diameter 16 may be between 2 and 6 mm. However, the varied diameters can cause an issue if the catheter 2 has a bevel 8. If the bevel 8 is too long, the part of the lumen 6 with a middle diameter 18 will be exposed. Therefore, the guide wire 4 may be unsecured within the lumen 6 and potential complications may arise. Therefore, the bevel 8 is preferably designed to ensure that only the part of the lumen 6 with an end diameter 16 is exposed. The length of the bevel 8 will depend on the specific design of the catheter 2.

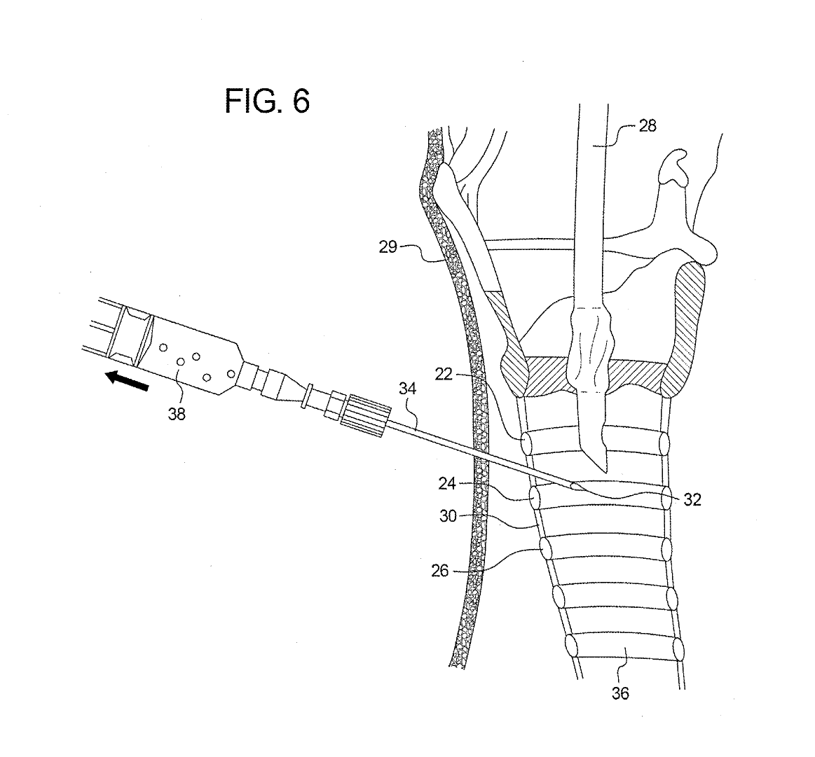

The beveled catheter has an additional function in a tracheostomy procedure, where the proper orientation of the bevel ensures the proper mating of the guide wire and catheter. FIGS. 6 through 10 show the tracheostomy procedure in detail.

Referring to FIG. 6, the tracheostomy tube (not pictured) is ideally placed between the first tracheal cartilage 22 and the second tracheal cartilage 24 or between the second tracheal cartilage 24 and the third tracheal cartilage 26. Generally, an endotracheal tube 28 is already in place to assist the patient in breathing during the procedure. The endotracheal tube 28 is raised above the first tracheal cartilage 22 to prevent interference with the tracheostomy procedure. Local anesthesia is then introduced to the area and a 1-1.5 centimeter vertical incision is made in the patient's skin 29. The skin 29 is then separated to provide unimpeded access to the tracheal wall 30.

Still referring to FIG. 6, a needle 32 is placed within a catheter 34 so that the point of the needle 32 extends outside of the catheter 34. The needle 32 is then used to puncture the tracheal wall 30 and access the trachea 36. Prior to puncturing the tracheal wall 30, a vacuum is applied to a syringe 38 attached to the needle 32 so that air bubbles in the syringe 38 will indicate when the needle 32 punctures the tracheal wall 30 and accesses the trachea 36. The syringe 38 and the needle 32 are then removed while the catheter 34 remains in the trachea 36.

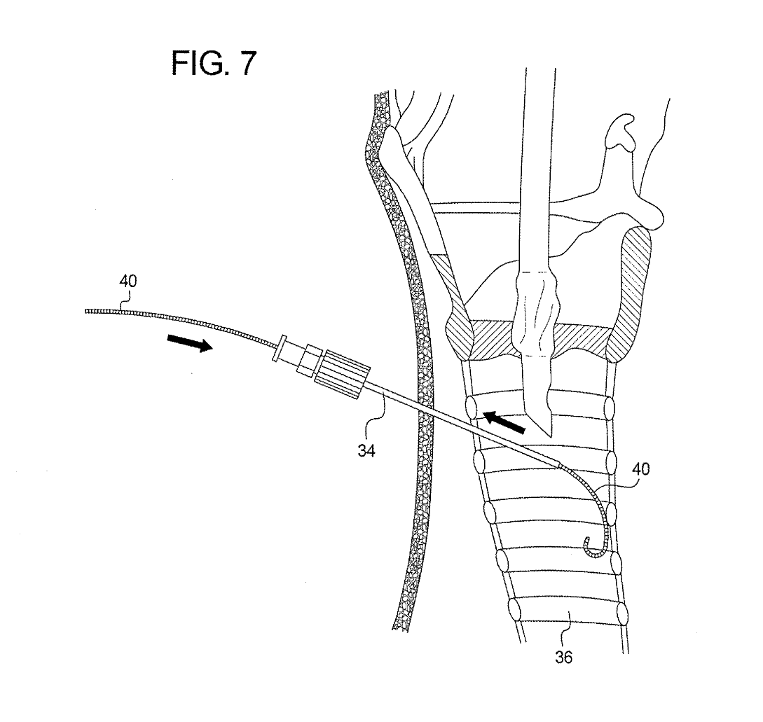

Now referring to FIG. 7, a guide wire 40 is fed through the catheter 34 into the trachea 36. The guide wire 40 is fed down the trachea 36 towards the lungs. Once the guide wire 40 is in place, the catheter 34 is removed, leaving the guide wire 40 in position, with part of the guide wire 40 within the trachea 36 and part of it outside of the patient and accessible to the clinician. Alternatively, the steps in FIGS. 6 and 7 can be performed without the catheter 34. Instead, the needle 32 can puncture the tracheal wall 30 without a catheter 34. The guide wire 40 is then fed through a lumen in the needle 32. Once the guide wire 40 is in place, the needle 32 is then removed from the trachea 36.

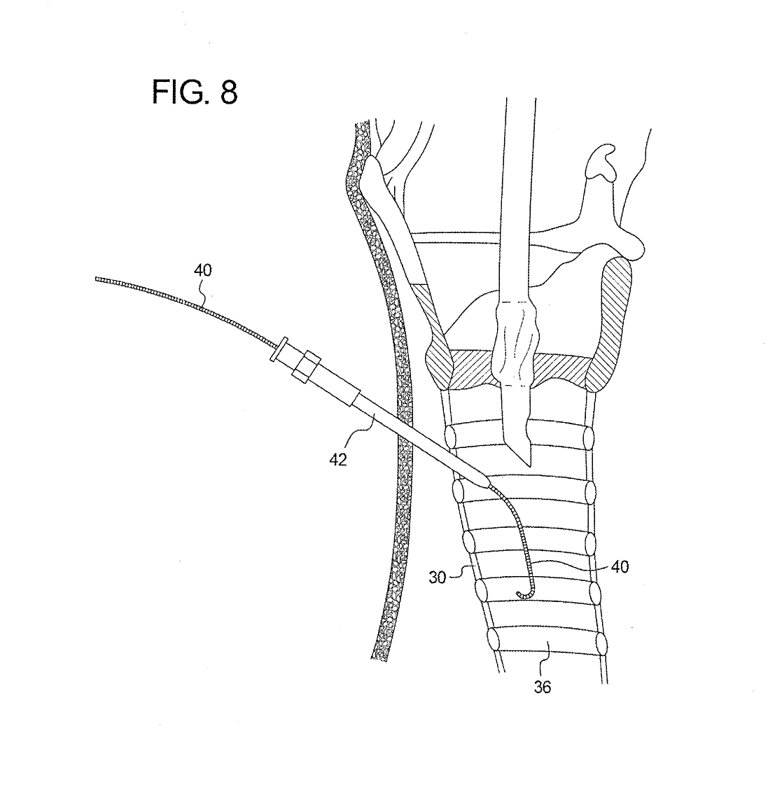

Referring to FIG. 8, an introducer dilator 42 with at least one lumen is then fed over the wire guide 40 and is slowly pushed through the tracheal wall 30. The introducer dilator 42 slowly dilates the hole in the tracheal wall 30. Once the hole is properly dilated, the introducer dilator 42 is then removed from the trachea 36 while the wire guide 40 remains in position.

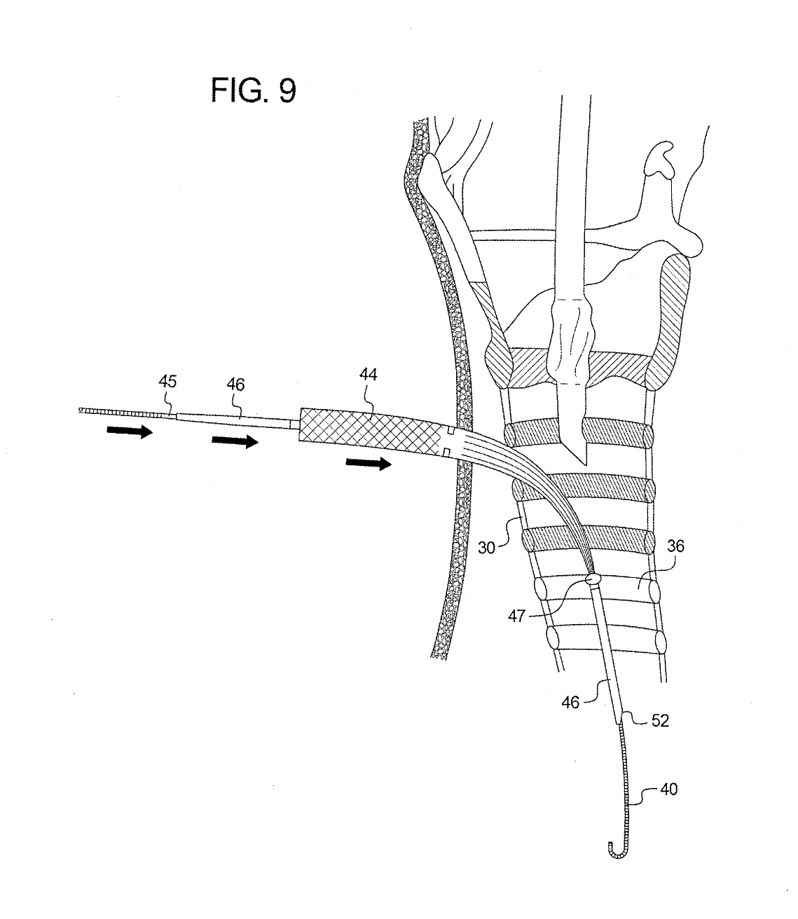

Now referring to FIG. 9, a curved dilator 44 is used in conjunction with a guiding catheter 46 to continue dilating the hole in the tracheal wall 30. The curved dilator 44 is ideally tapered to allow the hole in the tracheal wall 30 to be smoothly and gradually dilated and has a curve that is preset or fixed. The guiding catheter 46 can also have a preset, fixed curve that matches the fixed curve of the curved dilator 44. The matching fixed curves of the curved dilator 44 and the guiding catheter 46 prevent the guiding catheter 46 from freely rotating within the curved dilator 44. While the guiding catheter 46 may rotate if enough force is applied, the guiding catheter 46 tends to return to its original position when released due to the matching fixed curves.

Still referring to FIG. 9, the guiding catheter 46 is placed in a lumen of the curved dilator 44, and the curved dilator 44 is fed over the guiding catheter 46 until the distal end of the curved dilator 44 abuts the stop 47 on the guiding catheter 46. The stop 47 indicates that the curved dilator 44 and the guiding catheter 46 are properly positioned relative to each other and prevents the curved dilator 44 from sliding further down the distal end of the guiding catheter 46. Often, the guiding catheter 46 is provided already in position within the curved dilator 44. During the manufacturing process, the guiding catheter 46 may be inserted into the curved dilator 44, and then the assembly may be heated during the sterilization process. When the assembly is heated, the guiding catheter 46 takes the curved shape of the curved dilator 44. The curved dilator 44 and the guiding catheter 46 are then provided as one piece to clinicians.

Still referring to FIG. 9, the distal end of the guiding catheter 46 is fed over the proximal end of the guide wire 40 via a lumen in the guiding catheter 46. Together, the guiding catheter 46 and curved dilator 44 are fed into the trachea 36, following the path of the guide wire 40. Once the proximal end of the guiding catheter 46 is aligned with the position mark 45 on the guide wire 40, the curved dilator 44, guiding catheter 46, and guide wire 40 are fed into the trachea 36 simultaneously. Feeding the entire assembly into the trachea 36 together ensures that the distal end of the guiding catheter 46 does not advance beyond the distal end of the guide wire 40. During this process, the curved dilator 44 is pushed slowly into the trachea 36 until the hole in the tracheal wall 30 is properly dilated. Additionally, the curved dilator 44 is advanced and retracted several times to ensure proper dilating. The curved dilator 44 is then removed from the trachea 36. The guiding catheter 46 and the guide wire 40 remain in place.

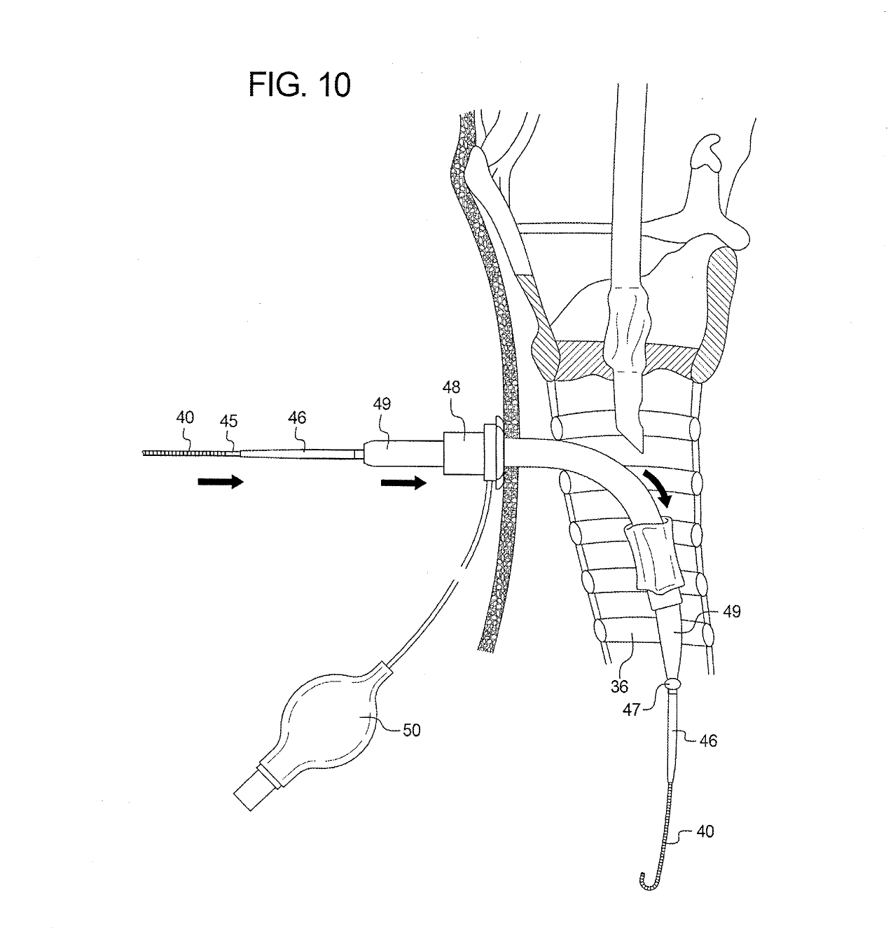

Looking at FIG. 10, the tracheostomy tube 48 is loaded onto an introducer 49. The introducer 49 and tracheostomy tube 48 are then fed onto the guiding catheter 46 until the distal end of the introducer 49 abuts the stop 47 on the guiding catheter 46. The stop 47 indicates that the introducer 49 and the guiding catheter 46 are properly positioned relative to each other and prevents the introducer 49 from sliding further down the distal end of the guiding catheter 46. The tracheostomy tube 48, introducer 49, and guiding catheter 46 are then fed over the guide wire 40 via a lumen in the introducer 49 into the trachea 36. Once the proximal end of the guiding catheter 46 is aligned with the position mark 45 on the guide wire 40, the introducer 49, tracheostomy tube 48, guiding catheter 46, and guide wire 40 are fed into the trachea simultaneously. Once the tracheostomy tube 48 is in place, the guide wire 40, guiding catheter 46, and introducer 49 are then removed from the trachea 36 while the tracheostomy tube 48 remains. A ventilator 50 can then be attached to the tracheostomy tube 48 to assist the patient's breathing.

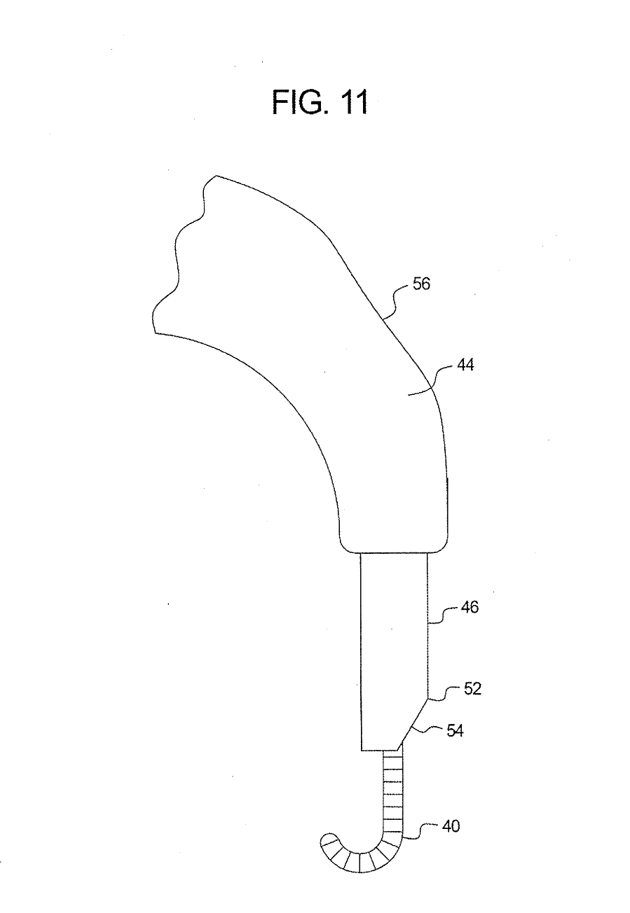

Now referring back to FIG. 9 in conjunction with FIG. 11, a bevel 52 may be on the end of the guiding catheter 46. FIG. 11 shows a detailed view of the bevel 52. The bevel 52 exposes part of the lumen 54 of the guiding catheter 46 along the side of the guiding catheter 46. The bevel 52 is oriented so that the exposed part of the lumen 54 is facing the side of the curved dilator 44 that is defined by a convex curve 56. The bevel 52 is oriented as described to prevent the guide wire 40 from pulling away from the guiding catheter 46 during the tracheostomy procedure. When the guiding catheter 46 is fed over the guide wire 40 into the trachea 36 the guide wire 40 will tend to slide along the inside surface of the lumen 54 on the concave side of the curved dilator 44. With the current orientation of the bevel 52, most of the lateral force applied by the guide wire 40 against the guiding catheter 46 will be directed toward the non-beveled side of the guiding catheter 46 and minimal force will be directed against the bevel 52. As a result, the guide wire 40 remains aligned with and supported by the guiding catheter 46. By contrast, if the bevel 52 was oriented on the concave side, the guide wire 40 may tend to pull away from the convex side of the guiding catheter 46 due to the weaker support provided by the bevel 52 and a gap may form on the convex side between the guide wire 40 and the guiding catheter 46. Additionally, because the preset, matching curves of the guiding catheter 46 and the curved dilator 44 prevent the guiding catheter 46 from freely rotating within the curved dilator 44, the bevel 52 remains properly oriented on the convex side of the curved dilator 44 throughout the procedure. While the guiding catheter 46 may rotate within the curved dilator 44 if enough force is applied, the guiding catheter 46 will tend to rotate back to its original position when the force is removed, thus ensuring the proper orientation of the bevel 52.

If the bevel was oriented differently, potential complications could arise during the tracheostomy procedure. For example, feeding the guiding catheter 46 and the curved dilator 44 over the guide wire 40 would be more difficult because the guiding catheter 46 would not smoothly follow the path of the guide wire 40. Therefore, there would be more friction between the guiding catheter 46 and the guide wire 40, thus creating more resistance. Also, the end of the guiding catheter 46 might jut out from the guide wire 40, increasing the risk of the guiding catheter 46 getting caught on the tracheal wall 30 and even causing damage to it.

In addition to the advantages of orienting the bevel this way for tracheostomy procedures, this same design could be utilized in other procedures that use a curved catheter or device in conjunction with a guide wire.

Additionally, a bevel can be used with other devices involved in tracheostomies. For example, the introducer dilator 42 of FIG. 8 can include a bevel to facilitate guide wire insertion. The introducer 49 of FIG. 10 can also include a bevel for the same reasons.

In another embodiment, the specialized bevel 8 of FIG. 4 may be placed on the end of the guiding catheter 46 of FIGS. 9 and 11. With this embodiment, the guide wire 40 can be easily inserted into the lumen 54, and the bevel 52 is properly oriented to create an efficient and effective tracheostomy procedure.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A dilator assembly, comprising:
a curved dilator comprising a proximal end, a distal end, a fixed curvature of the curved dilator being defined by a concave side and a convex side, the curved dilator further comprising at least one lumen curved along a portion thereof;
a guiding catheter comprising a proximal end and a distal end, the guiding catheter further comprising at least one lumen;
wherein the guiding catheter is disposed within the at least one lumen of the curved dilator and is curved along the curved portion of the at least one lumen of the curved dilator, with the distal end of the guiding catheter extending past the distal end of the curved dilator;
wherein the guiding catheter further comprises a bevel on the distal end of the guiding catheter with an exposed area of the at least one lumen of the guiding catheter extending proximally from the distal end of the guiding catheter, the bevel oriented so that the exposed area of the at least one lumen of the guiding catheter faces toward the convex side of the curved dilator; and
a guide wire comprising a proximal end and a distal end, wherein the guide wire is disposed within the at least one lumen of the guiding catheter and is curved along the curved portion of the at least one lumen of the curved dilator, with the distal end of the guide wire extending past the distal end of the guiding catheter.

2. The dilator assembly of claim 1, wherein:
the guiding catheter comprises a fixed curvature corresponding to the fixed curvature of the curved dilator.

3. The dilator assembly of claim 1, wherein:
the curved dilator is a tracheostomy dilator.

4. The dilator assembly of claim 1, wherein:
the curved dilator comprises a taper extending from a smaller profile near the distal end and a larger profile away from the distal end.

5. The dilator assembly of claim 1, wherein:
the proximal end of the guiding catheter extends past the proximal end of the curved dilator.

6. The dilator assembly of claim 1, wherein:
the proximal end of the guide wire extends past the proximal end of the guiding catheter.

7. The dilator assembly of claim 1, wherein:
the guiding catheter further comprises a wall surrounding the at least one lumen, an outer diameter extending along a majority of a distal portion of the guiding catheter, and an outer surface;
wherein the bevel further comprises a distal end, a proximal end, and an intermediate point, wherein the distal end of the bevel is in line with the distal end of the catheter, a first portion of the bevel extending from the distal end to the intermediate point and a second portion of the bevel extending from the intermediate point to the proximal end, wherein along the first portion, the wall of the guiding catheter extends 160-225 degrees circumferentially around the at least one lumen without extending around a remaining circumferential area of the at least one lumen, and along the second portion, the wall gradually slopes from the intermediate point to a uniform outer circumference of the guiding catheter at the proximal end of the bevel, wherein a longitudinal distance of the first portion is between 1 and 5 times the outer diameter of the guiding catheter.

8. The dilator assembly of claim 7, wherein:
the curved dilator is a tracheostomy dilator, the tracheostomy dilator comprising a taper extending from a smaller profile near the distal end and a larger profile away from the distal end, wherein the guiding catheter comprises a fixed curvature corresponding to the fixed curvature of the curved dilator.

9. A catheter system, comprising:
a catheter comprising at least one lumen, a wall surrounding the at least one lumen, an outer diameter extending along a majority of a distal portion of the catheter, an outer surface, and a bevel on at least one end of the catheter; and
a guide wire oriented within the at least one lumen of the catheter;
wherein the bevel comprises a distal end, a proximal end, and an intermediate point, wherein the distal end of the bevel is in line with the at least one end of the catheter, a first portion of the bevel extending from the distal end to the intermediate point and a second portion of the bevel extending from the intermediate point to the proximal end, wherein along the first portion, the wall of the catheter extends 160-225 degrees circumferentially around the at least one lumen without extending around a remaining circumferential area of the at least one lumen, and along the second portion, the wall gradually slopes from the intermediate point to a uniform outer circumference of the catheter at the proximal end of the bevel, wherein a longitudinal distance of the first portion is between 1 and 5 times the outer diameter of the catheter.

10. The catheter system of claim 9, wherein:
the at least one end of the catheter comprises a taper extending from a smaller profile near the at least one end of the catheter and a larger profile away from the at least one end of the catheter.

11. The catheter system of claim 9, wherein:
the gradual slope of the second portion has a concave arc shape.

12. The catheter system of claim 9, wherein:
the first and second portions comprise an outer edge, the outer edge comprising a fillet.

13. The catheter system of claim 9, wherein:
the wall of the catheter extends around the same circumferential area along the first portion.

14. The catheter system of claim 9, wherein:
the longitudinal distance of the first portion of the bevel is between 1 and 3 times the outer diameter of the catheter.

15. The catheter system of claim 9, wherein:
the at least one lumen comprises a first portion comprising a first inner diameter and a second portion comprising a second inner diameter, the first portion extending proximally from the at least one end of the catheter to a transition point, and the second portion extending proximally from the transition point; wherein the first inner diameter is smaller than the second inner diameter.

16. The catheter system of claim 15, wherein:
the bevel does not expose the second portion of the at least one lumen.

17. The catheter system of claim 16, wherein:
the longitudinal distance of the first portion of the bevel is between 1 and 3 times the outer diameter of the catheter.

18. The catheter system of claim 9, wherein:
the longitudinal distance of the second portion of the bevel is between 1 and 5 times the outer diameter of the catheter.

19. The catheter system of claim 18, wherein:
the longitudinal distance of the second portion of the bevel is between 1 and 2 times the outer diameter of the catheter.

20. The catheter system of claim 17, wherein:
the longitudinal distance of the second portion of the bevel is between 1 and 2 times the outer diameter of the catheter, the gradual slope of the second portion comprising a concave arc shape, wherein the first and second portions comprise an outer edge, the outer edge comprising a fillet, wherein the wall of the catheter extends around the same circumferential area along the first portion.
